(19) United States
(12) Patent Application Publication
(10) Pub. No.: US 2007/0213505 A1
(43) Pub. Date: Sep. 13, 2007

(54) Solution Synthesis of Peptide Cell Growth Stimulators

(76) Inventors: David Epstein, Philadelphia, PA (US); Marian F. Kruszynski, King of Prussia, PA (US); Christopher Marsh, Audubon, PA (US); Albert Schmidt, Abington, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/683,585

(22) Filed: Mar. 8, 2007

Related U.S. Application Data
(60) Provisional application No. 60/780,101, filed on Mar. 8, 2006.

(51) Int. Cl. C07K 5/08; C07K 1/02

(52) U.S. Cl. 530/331; 530/333

(57) ABSTRACT

A solution phase synthetic method for preparing basic tripeptides of the formula Gly-Xaa-Gly-X which have in various biological properties such as stimulating protein production when used as additives in a bioreactor. The basic tripeptides of the invention may be produced on gram or kilogram scale.

### CROSS REFERENCE TO RELATED APPLICATIONS

This invention claims priority to U.S. Provisional Application Ser. No. 60/780,101, filed 08 Mar. 2006, the entire contents of which is incorporated herein by reference.

### BACKGROUND OF THE INVENTION

1. Field of the Invention

The invention relates generally to the synthesis of peptides useful as cell growth stimulators. More particularly, the invention relates to the synthesis of short cationic peptides comprising a final one-step global deprotection of benzyloxycarbonyl protected peptides by suitable methods such as catalytic hydrogenation. The invention more particularly relates to methods of preparing the tripeptide cell growth stimulator, Gly-Lys-Gly, using a facile method wherein a benzyloxycarbonyl group is used for protection of both the N^{α}-amino group of glycine and the N^{ε}-amino side chain group of lysine and so that the final product is obtained in a single global deprotection step under neutral conditions using suitable deprotection methods.

2. Description of the Related Art

The need for production capacity and of therapeutic proteins using large-scale animal cell cultures continues to increase. Further, the need for ever more carefully controlled processes which eliminate the requirement for undefined and potentially contaminated additives such as animal byproducts (serum, tissue extracts) or plant-derived components such as soy extract or partially hydrolyzed proteins is mandated. However, the cost of manufacturing therapeutic protein products depends on the volumetric productivity achievable in bioreactor technology which, in turn, is dependent on inexpensive and available forms of growth enhancing supplements to sugar based growth medium.

Peptones, traditional components of microbial fermentation broth, were recognized more than 25 years ago as useful in the culture of mammalian cells (Mizrahi, A. Biotechnol. Bioeng. 19: 1557-1561, 1977). The prevalent opinion on their mode of action is as a rich and inexpensive source of amino acids needed for cell mass synthesis and for the synthesis of secreted proteins (Nyberg, G. B. et al., Biotechnol. Bioeng. 62: 324-335, 1999). Alternatively, amino acids and small peptides have been used as growth stimulating agents in bioreactors for cell culture.

These compounds may act as anti-aptotic agents for hybridoma cultures (Franek, F. and Sramkova, K. Cytotechnology 21 (1): 81-89, 1996) or enhance productivity of mammalian cell cultures and suppress apoptosis (Franek, F. Biotechnol. Prog. 19: 169-174, 2006 and WO01/46220). In these studies, the authors identified the tripeptide gly-lys-gly as particularly potent in the later nonnutrient roles among various peptides comprising three to five amino acid residues.

Synthesis of biologically active peptides can be achieved using several chemical, biochemical as well as recombinant strategies. However, for scale up, the route of synthesis for each peptide requires a different approach. The guidelines for scale up can be related to the size of peptide and the amount of product needed. In general, the best approach for production of large quantities of short peptides is solution synthesis. The cost of selected tri- or tetrapeptides synthesized in quantities of kilograms for large-scale cultures must be low if they are to be used in manufacturing processes.

Liquid phase methods (often referred to as solution phase methods) of synthesis carry out all reactions in a homogeneous phase. Successive amino acids are coupled in solution until the desired peptide material is formed. During synthesis, successive intermediate peptides are purified by precipitation and/or washes. Peptides and amino acids from which peptides are synthesized tend to have reactive side groups as well as reactive terminal ends. When synthesizing a peptide, it is important that the amino group on one peptide react with the carboxyl group on another peptide. Undesired reactions at side groups or at the wrong terminal end of a reactant produces undesirable by-products, sometimes in significant quantities. These can seriously impair yield or even ruin the product being synthesized from a practical perspective. To minimize side reactions, it is conventional practice to appropriately mask reactive side groups and terminal ends of reactants to help ensure that the desired reaction occurs.

The major advantage of solution peptide synthesis is the availability of a multitude of coupling methods, a wide variety of protecting groups, opportunities for intermediate purification and the potential for linear scale up. In planning solution synthesis the strategy considerations include: (1) selection of main chain and side chain protective groups; (2) choice of activation method; (3) deprotection methods; (4) selection of segments in efforts to minimize recemization during segment condensation; and (5) solubility considerations. Conventional solution peptide synthesis is cumbersome and time consuming, but it allows purification of fully protected intermediates. This is an advantage over solid phase peptide synthesis (SPPS), in which case the final product has to be isolated from a mixture of similar fragments

Based on the need for defined growth and production enhancing cell culture additives for large scale bioreactors, and the identification of specific growth enhancing tripeptides, there is a need in the art for reproducible cost effective methods of solution synthesis of these peptides.

### SUMMARY OF THE INVENTION

The present invention provides a method of solution phase peptide synthesis of producing a basic tripeptide of the formula Gly-Xaa-Gly-X wherein Xaa is an alpha amino acid comprising a basic side chain; Gly-X is glycine or a glycyl-ester or glycinamide. The method of the invention uses a benzyloxycarbonyl moiety (A) for protection of the basic sidegroup or sidegroups of Xaa and at the alpha amine of

glycine in the final coupling step. An orthogonal protecting group (B), such as Boc, protects the alpha of the Xaa in the initial coupling and the orthogonal protecting group is cleavable using conditions not suitable for removing the benzyloxycarbonyl moiety. The first step of the synthesis is the reaction of the protected basic Xaa species (B-Xaa(A)ₙ, where n=1 for monoamine sidechains and 2 for guanidino-containing sidechains) with glycine or a glycine derivative (Gly or Gly-X) to get a compound of the formula B-Xaa(A)ₙ-Gly or B-Xaa(A)ₙ-Gly-X followed by removal of the orthogonal alpha-amine protecting group. The second coupling is achieved by reacting the deprotected compound with carbobenzyloxy protected Gly (Z-Gly) to get a compound of formula III, N^{α}-A-Gly-Xaa(A)ₙ-Gly or N^{α}-A-Glycine-L-Xaa(A)ₙ-Gly-X. The final product is produced by the single step removal of all benzyloxycarbonyl groups using hydrogenation. The resulting tripeptide or tripeptide amide or ester may be obtained by precipitation.

In another aspect, the invention relates to the novel intermediate of the formula III, N^{α}-A-Gly- Xaa(A)ₙ-Gly or N^{α}-A-Glycine-L-Xaa(A)ₙ-Gly-X useful for preparation of the tripeptide cell growth stimulator of the formula Gly-Xaa-Gly-X.

In another aspect of the invention, a peptide synthesized by the method of the invention is used in a bioreactor either as a single species or in combination with other peptides of a defined nature for the purpose of enhancing bioreactor productivity and decreasing the extent of apoptotic death of the cells of the bioreactor.

### BRIEF DESCRIPTION OF THE SEVERAL VIEWS OF THE DRAWING

FIG. 1 shows a schematic depiction of the solution peptide synthetic method of the invention for the exemplary basic tripeptide, Gly-Lys-Gly.

FIG. 2 shows a tracing from a nanoelectrospray (NanoES MS) of the final product Gly-Lys-Gly (Batch D-5) giving the expected at a MW=260.26 Da [MWₜₕₑₒ=260.30 Da] with minor contaminants at m/z 226.2 Da, 318.3 Da, and 389.4 Da [Calculated mol. weight for cylco(Lys-Gly): 185.22 Da; Calculated mol. weight for Lys-Gly: 203.24 Da].

FIG. 3 shows a tracing from a capillary electrophoresis analysis of Gly-Lys-Gly (Batch GKG-2) (Beckman Coulter P/ACE™ MDQ instrument using a photodiode array (PDA) detector, bare fused-silica capillary (75 μm ID×50 cm) and 50 mmol phosphate buffer, pH 2.5. and 25 kV, normal polarity voltage used for over 30 min at 25° C.

FIG. 4 is a plot of cell viability during six days of culture in the presence and absence of Gly-Lys-Gly.

FIG. 5 is a plot showing three parameters of cell viability and productivity in the presence and absence of Gly-Lys-Gly or soy hydrolysate measured on the sixth day of culture; specific productivity expressed as pg or product produced per cell per day (pg/c/d), the titer of antibody in the cell supernatant expressed as mg/L, and integral viable cell concentration (IVCC).

### DETAILED DESCRIPTION OF THE INVENTION

Abbreviations

AcOEt ethyl acetate; AcOH acetic acid; AOP 7-azabenzotriazol-1-yloxy-tris(dimethylamino)phosphonium hexafluorophosphate; Arg L-arginine; Boc tert-butoxycarbonyl; BOP benzotriazol-1-yloxy-tris(dimethylamino)phosphonium hexafluorophosphate; Bzl benzyl; CE capillary electrophoresis; DCC N,N′-dicyclohexylcarbodiimide; DCM dichloromethane; DIC N,N′-diisopropylcarbodiimide; DMF N,N-dimethylformamide; ESI MS electrospray mass spectrometry; EtOH ethanol; Gly glycine; HATU N-[(dimethylamino)(1H-1,2,3-triazolo[4,5-b]pyridin-yl)methylene]-N-methylmethanaminiumhexafluorophosphate N-oxide; HBPyU O-(benzotriazol- 1-yl)-N,N,N′N′-bis(tetramethylene)uranium hexafluorophosphate HBTU N-[1 H-benzotriazol-1-yl)(dimethylamino)methylene]-N-methylmethanaminium hexafluorophosphate N-oxide; His L-histidine; HOAt 7-aza-1,2,3-benzotriazol-1-ol; HOBt 1,2,3-benzotriazol-1-ol; HOBt x H₂O 1,2,3-benzotriazol-1-ol monohydrate; HPLC high-performance liquid chromatography; Lys L-lysine; Mab monoclonal antibody; MgSO₄ magnesium sulfate; NMM N-methylmorpholine; OBzl benzyloxy; ODhbt 4-oxo-3,4-dihydro- 1,2,3-benzotriazin-3-yl; OPfp pentafluorophenyl; Osu succinimidoxy; Pd/C palladium/activated carbon; PyAOP 7-azabenzotriazol-1-yloxy-tripyrrolidinophosphonium hexafluorophosphate; PyBOP benzotriazol- 1-yloxy-tripyrrolidinophosphonium hexafluorophosphate; R_{f} retention factor (TLC); SELDI MS surface enhanced laser desorption ionization mass spectrometry; TFA trifluoroacetic acid; Tfa trifluoroacetyl; TLC thin-layer chromatography; Z benzyloxycarbonyl; Z(Cl) 4-chlorobenzyloxycarbonyl; Z(2-Cl) 2-chlorobenzyloxycarbonyl.

Note: The coupling reagents HBTU, HATU, and their derivatives have been named in this list using the aminium nomenclature which were originally thought to have the uronium structure but instead have the isomeric N-oxide structure and which unwieldy corresponding name does not conform logically with the acronym. Thus, the acronyms remain in use.

Definitions

All amino acids, unless otherwise specified, are taken be the naturally occurring L-isomeric form of the alpha-amino acid. Peptides, unless otherwise specified, are taken to comprise the naturally occurring L-amino acid forms. While the L-amino acid forms of basic amino acids are most commonly used, the D-forms are also amenable to the synthetic method of the present invention.

Solution Synthesis of Peptides

The benzyloxycarbonyl strategy of the present invention used in solution synthesis of a tripeptide compound Gly-Xaa-Gly-X (IV) via a protected intermediate wherein at least all basic groups are protected, N^{α}-U-Gly-Xaa(A)ₙ-Gly-X (III), represents the cost effective and simple method for producing short peptides containing a the basic amino acid, lysine, omithine, histidine, arginine or homoarginine, α, γ-diaminobutyric acid, or α, β-diaminopropionic acid.

Compounds and intermediates used and generated by the process of the invention are described as follows:
a fully protected basic amino acid N^{α}-B-Xaa(A)ₙ where
Xaa is a basic amino acid selected from the group consisting of Lys, Orn, His, Arg or Homoarg, Dab, or Dap;
A is a benzyloxycarbonyl protecting group selected from the group consisting of Z, Z(Cl), and Z(2-Cl); and n is 1 for basic amino acids have a single amine sidegroup and 2 for amino acids containing a guanidine group;
B is a protecting group which is unstable under conditions wherein A is stable such as acid and base catalyzed hydrolysis and B may be selected from the group consisting of Boc, Bpoc, 1-Adoc, Ddz, Fmoc, Nsc, and Msc;
   a glycine, carboxy protected glycine such as Gly-OBzl or O-Aryl, or a glycine derivatized or modified at the carboxylic group represented by Gly-X wherein X may be OH, OR₁ or —N(R₁ R₂) where R₁ and R₂ are independently H, small alkyl, acyl, fatty acyl, or aryl;
      a basic protected dipeptide intermediate of the formula
      N^{α}-B-Xaa(A)n-Gly-X I
      where Xaa, A, R1, and n have the meanings described above;
   a deprotected dipeptide of the formula
      Xaa(A)ₙ-Gly-X II
      where Xaa, A, R1, and n have the meanings described above;
   a basic protected tripeptide intermediate of the formula
      N^{α}-A-Gly-Xaa(A)ₙ-Gly-X III
      where Xaa, A, R1, and n have the meanings described above; and
   a final deprotected product tripeptide of the formula
      Gly-Xaa-Gly-X IV
      where Xaa, A, X, and n have the meanings described above except that where X cannot be a O-Bzl or O-Aryl.

Solution peptide synthesis allows purification of fully protected intermediate dipeptides of formula (I), such as Boc-Lys(Z)-Gly-OBzl, and the protected tripeptides of formula III, such as N-A-Lys(A)-Gly-OBzl after simple precipitation from water. The method of the invention provides a final, one-step deprotection of precursor under neutral conditions. This is an advantage over solid phase peptide synthesis (SPPS), in which case the final product has to be isolated from a mixture of similar fragments. The synthetic method of the invention provides a strategy allowing scale-up of the solution synthesis of basic peptides, including Gly-Lys-Gly, to kilogram quantity. The cost of a selected tri- or tetrapeptides synthesized in quantities of hundreds of kilograms may go down 100-500 times in comparison with current catalog prices.

The most commonly employed methods for peptide bond formation in solution include: the carbodiimide method (DCC, DIC), symmetric or mixed anhydrides, active esters (OPfp, Odhbt, OSu), phosphonium salts (BOP, PyBOP, AOP, PyAOP) and uronium/guanidinium-mediated salt build around processes using HOBt and HAOt (HBTU, HATU, HBPyU, etc).

Glycine esters without protonated amino function undergo spontaneously cyclization to form stable 2,5-diketopiperazine derivatives. Therefore, selection of the Gly reagent for the first coupling can be selected from carboxy protected reagents and their salts or free base as shown in Table 1. Where the desired tripeptide produced by the method of the invention is to have a free carboxyl terminus, the glycine ester must be selected such that the final deprotection reaction also hydrolyzes the glycine ester selected for the first coupling reaction.

TABLE 1

| Species | Structure |
|---|---|
| Glycine benzyl ester tosylate | H-Gly-OBzl × Tos |
| Glycine benzyl ester hydrochloride | H-Gly-OBzl × HCl |
| Glycine ethyl ester hydrochloride | H-Gly-OEt × HCl |
| Glycine methyl ester hydrochloride | H-Gly-OMe × HCl |
| Glycine tert-Butyl ester hydrochloride | H-Gly-OBu^{t} × HCl |
| Glycine amide hydrochloride | H-Gly-NH₂ × HCl |
| Glycine methylamide hydrochloride | H-Gly-NHMe × HCl |
| Glycine hydrochloride | H-Gly-OH × HCl |
| Glycine free base | H-Gly-OH |
| Glycine 7-amido-4-methylcoumarin | H-Gly-AMC × HBr |
| hydrobromide | |
| Glycine p-nitroanilide hydrochloride | H-Gly-pNA × HCl |
| Glycine β-Naphthylamide hydrochloride | H-Gly-βNA × HCl |

HBTU reacts exclusively with carboxylate salts (R—COO⁻); mixtures of HBTU and a carboxylic acid (R—COOH) remain stable. Examples related to the present invention are Boc-Lys(Z)-COOH+NMM or Boc-Lys(Z)-COO—+NMMH+. This procedure eliminates the requirement for a separate neutralization step (Tos x Gly-OBzl, Tos x Gly-NH₂ or TFA x Lys(Z)-Gly-OBzl, TFA x Lys(Z)-Gly-NH₂) saving time and minimizing diketopiperazine formation. Three equivalents of base (DIEA or NMM) are necessary to neutralize the carboxylic acid, the amine salt, and the acidic hydroxybenzotriazole.

When using HBTU, the reaction mixture has to be kept near basic pH in order to insure a fast coupling. Under such conditions, the coupling rate is so high that racemization is negligible using urethane-protected amino acid couplings and fairly low in segment coupling. The excess of acid and “onium” salt (HBTU) is typically 1.1 molar equivalent in solution synthesis. In solid phase, excess from 1.5-3 equivalents are commonly used. The byproducts are water- and DCM-soluble that allows purification of product by precipitation by water (in solution synthesis).

For the correct assembly of peptide sequence, the N^{α}-amino protecting group should be specifically cleavable while leaving the side-chain protecting groups intact (“orthogonal” protection). Other reactive functional groups that require mandatory protection are side-chain amine (Lys), carboxylic acid (Asp, Glu), and the thiol (Cys) groups; protection of hydroxyl (Ser, Thr, Tyr), guanidine (Arg), imidazole (His), and the indole (Trp) groups while optional, is often preferred for minimizing the formation of side products. The overall selection of protecting groups is dictated by the synthetic strategy.

The deprotection method used during stepwise assembly of peptide chain and for the final deblocking of all functional groups to yield the free peptide is determined by the synthetic strategy. In the present invention, suitable orthogonal amine protecting groups may be selected from those given in Table 2 below and deprotection may be affected for the group using conditions amenable to hydrolysis of the group as specified in Table 2.

TABLE 2

| Amine | |
|---|---|
| Protecting | |
| Group (B) | Suitable Deprotection conditions |
| Boc | TFA, TFA/DCM (1:1), HCl/dioxane, HCl/AcOH, |
| | HCl/AcOEt, 70% TFA/H₂O, TFA/AcOH, AlCl₃, TosOH × |
| | H₂O, BF₃ × OEt₂/AcOH, TMSI. |
| Bpoc | 0.5% TFA/DCM (weak acid) |
| 1-Adoc | TFA |
| Ddz | diluted TFA |
| Fmoc | basic, e.g. piperidine/DMF, morpholine, dimethylamine |
| | in solution, Dbu in solution |
| Nsc | basic, e.g. 20% piperidine/DMF |
| Msc | DMF/MeOH/4 M NaOH |

For the final deprotection, several options for deprotection reaction processes are shown below and include (a) catalytic hydrogenolysis using molecular hydrogen and palladium (H₂/Pd) or catalytic transfer hydrogenolysis; (b) reduction with metallic sodium in liquid ammonia and (c) or reaction with strong acids using halogenated acids or hydrohalogenic acids, e.g. hydrobromic acid in acetic acid (HBr/AcOH) or liquid HF, BBr₃/DCM, TFA/thioanisole and sulfonic acids.

Of these, cleavage of protecting groups under neutral conditions is highly desirable because such procedures would: (a) be free from racemization; (b) leave acid sensitive amino acids intact; (c) be useful particularly in the synthesis of acid/base sensitive biologically active peptides; and (d) eliminate side reactions that are commonly observed in acidolysis and base treatments. Two such methods, catalytic hydrogenation (Schlatter, J. M. et al. Tetrahedron Lett. 33: 2851-2852 (1977); Rylander, P. N. Catalytic Hydrogenation in Organic Syntheses. New York: Academic Press, 1979; Kieboom, A. P. G.; van Rantwijk, F. In Hydrogenation and Hydrogenolysis in Synthetic Organic Chemistry, Delft University Press, 1977) and catalytic transfer hydrogenation (Khan, S. A., and Sivanandaiah, K. M. Synthesis, 1978: 750-75; Anwer, M. K., and Spatola, A. F. Tetrahedron Lett. 22: 4369-4372, 1981; Anantharamaiah, G. M., and Sivanandaiah, K. M. J. Chem. Soc. Perkin Trans. 1 5: 490-491, 1977) have become popular for the cleavage of benzyl esters, benzyl ethers and benzyloxycarbonyl protecting groups. The only limitation being the intolerance of palladium catalysts to the presence of divalent sulfur. While hydrogenation with gaseous hydrogen is typically performed under high pressure (Parr apparatus), transfer hydrogenation is typically performed under ambient pressure. The transfer hydrogenation procedure utilizes simple organic molecules (cyclohexene, cycloxexadiene, formic acid) or inorganic compounds (phosphinic acid and its salts, hydrazine) as in situ source of hydrogen.

Thus, the glycine species used in the second coupling step should be protected at the amine with a group capable of being released by hydrogenolysis under neutral conditions. Suitable reagents are shown in Table 3.

TABLE 3

| Species/Structure | Deprotection Conditions |
|---|---|
| Z-Gly-OH | H₂/Pd, HBr/AcOH |
| Z(2-Cl)-Gly-OH | H₂/Pd, HBr/AcOH |
| Bzl-Gly-OH | H₂/Pd |
| (Bzl)2Gly-OH | H₂/Pd |
| Trt-Gly-OH | H₂/Pd, 1 M HCl/EtOH, HOBt/TFE |
| PhFl-Gly-OH | H₂/Pd, TFA, aq. MeCN |

Cell Cultures

Soy hydrolysates are extremely complex mixtures of peptides. The identity of the active component(s) in soy hydrolysate is unknown. Although soy hydrolysate supplementation can significantly enhance antibody production, there are frequent problems associated with the use of soy hydrolysate. These are: a) lot-to-lot variability of its composition due to variability of quality and performance of hydrolyzing enzymes; b) potential contamination of hydrolysate with animal components during or prior to manufacturing; c) carryover of active hydrolyzing enzymes in the hydrolyzate that can adversely affect stability and product quality of the protein products; and d) contamination of hydrolysates with agricultural chemicals such as pesticides and herbicides. Some of these problems were found to be severe enough during the development of recombinant proteins that the use of soy hydrolysate was discontinued.

Whenever the culture conditions in a bioreactor start to deviate significantly from the physiological range, cultured animal cells tend to reduce their viable population size by setting into action the process of programmed cell death called apoptosis. The potential of cultured cell lines to reduce their population size by apoptotic death prevents the collapse of the culture that would inevitably follow a total exhaustion of nutrients and enables cells to survive as smaller population under conditions of starvation.

Apoptotic suicide starts to reduce the population long before cell nutrients are exhausted. This feature of apoptotic death allows for application of signals that persuade the starving cells to postpone their suicide. Survival-signal molecules do not primarily promote the rate of cell growth, but they suppress the rate of apoptotic death, even if the levels of nutrients are critically low. The idea of survival-signal action of peptides resulted from studies by Franek at al. (Franek, F., Sramkova, K.

Cytotechnology 21: 81-89, 1996; Franek, F. BioProcess Int. 2004: 45-82; Franek, F. et all. Biotechnol. Prog. 19: 169-174, 2003). Some synthetic peptides improved all culture parameters, the other group promoted solely the growth of the cultures, and other peptides enhanced the product yield (Franek, F. BioProcess Int. 2004: 48-52). However, the growth-stimulating and the production-enhancing effects of peptides require peptide concentrations at least in the millimolar range (Franek, F. supra).

The host cell can optionally be at least one selected from E. Coli, COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof. Also provided is a method for producing at least one peptide or antibody of the invention, comprising translating the peptide or antibody encoding nucleic acid under conditions in vitro, in vivo or in situ, such that the peptide or antibody is expressed in detectable or recoverable amounts.

The nucleic acids encoding the therapeutic proteins produced by the cell culture methods of this invention can be obtained in several ways well known in the art. In one aspect, the therapeutic proteins are antibodies. Nucleic acids encoding antibodies are conveniently obtained from hybridomas derived from the fusion of a B-cell lineage with a fusion partner or cloned from other host cells by any of the techniques well known in the art, see, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001), each entirely incorporated herein by reference.

The availability of pure growth- and/or production-promoting synthetic peptides would represent a challenge for novel media formulations, meeting the most severe criteria of biological safety. The presentation of a survival-signal to cultured cells will open the way to complex feeding strategies exploiting the extended lifespan of the cultures for continuing product synthesis.

Other Uses of Peptides

In addition to the nonnutritive role in bioreactor productivity, peptides such as gly-lys-gly and related peptides have been found to have other biological properties. For example, lysine containing peptides may be anti-bacterial by virtue of their ability to compete for lysine uptake by lysine-dependent species of Pseudomonas. Small peptides of two to ten amino acids, may also inhibit viral infectivity by binding to a protein involved in capsomere organization and capsid assembly of HIV-1, HIV-2, and SIV thereby preventing proper capsid assembly and therefore the ability of the virus to infect cells (U.S. Pat. No. 6,258,932). The small peptides which are active in viral functionality are amides of the normal carboxy terminated tripeptides and include the cationic peptides: gly-pro-gly-NH₂, gly-lys-gly-NH₂, and arg-gln-gly-NH₂. Amides of small cationic peptides are amenable to the methods of the invention.

While having described the invention in general terms, the embodiments of the invention will be further disclosed in the following examples.

### EXAMPLE 1

### Preparation of N^{α}-Butyloxycarbonyl-L-Lysine (N^{ε}-Benzyloxycarbonyl)-Glydine Benzyl Ester (Boc-Lys(Z)-Gly-OBzl)

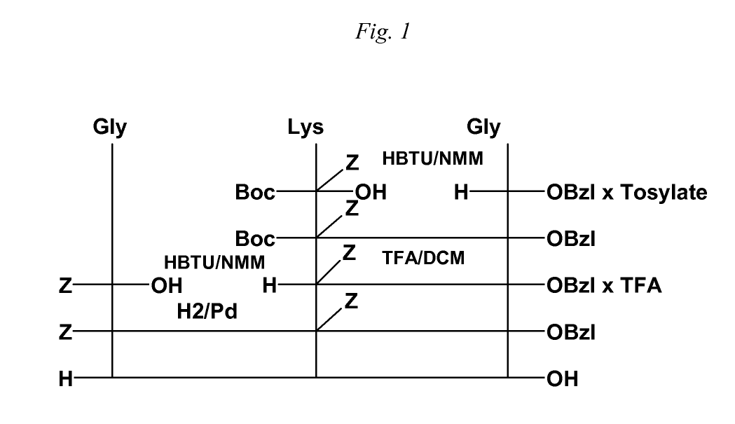

The preparation of the tripeptide Gly-L-Lys-Gly was performed in a stepwise manner as shown in the accepted conventional notation shown in FIG. 1, and includes a) a first coupling b) deprotection c) second coupling d) deprotection and e) purification. The first coupling step of the method for cationic tripeptide synthesis yields a protected dipeptide exemplary of the compounds of Formula I which is Boc-Lys(Z)-Gly-Obzl. The coupling is detailed herein.

H-Gly-OBzl x Tos (3.374 g, 10 mmol), HOBt x H2O (1.531 g, 10 mmol), Boc-Lys(Z)-OH (3.804 g, 10 mmol), and HBTU (3.7925 g, 10 mmol) were dissolved in DMF (35 mL), stirred and cooled in an ice-bath. NMM (4.16 mL, 37.8 mmol) was added and stirring was continued for one hour at 0° C. and overnight (18 hours) at ambient temperature (pH was controlled to keep 7.5-8 as indicated by moistened pH paper). The progress of the reaction was monitored by TLC using chloroform:methanol (CM)=9:1 (v/v); detection with ninhydrin prior to addition of NMM and at t=2.5 h, and after 18 h when the product was an oil, precipitated with water.

Water (600 mL) was added to reaction mixture. The precipitated oil was separated, washed with water (2×50 mL), dissolved in ethyl acetate (175 mL), dried over anhydrous MgSO4. After filtration, solvent was evaporated in vacuo and designated Batch A-1. The yield was 4.61 g (87.3%), C₂₈H₃₇N₃O₇, MW 527.65 Da, confirmed by SELDI-MS to be 548.6 Da [M +Na]+, and by TLC the R_{F}=0.72 (single spot using in chloroform:methanol=9:1 (v/v) using ninhydrin or iodine detection.

The synthesis of protected dipeptide (Boc-Lys(Z)-Gly-Obzl) was repeated on a larger scale (33.9 mM) which yielded 18.34 g of product.

### EXAMPLE 2

### Deprotected Intermediate L-Lysine(N^{ε}-Benzyloxy Carbonyl)-Glycine Benzyl Ester Trifluoroacetate (H-Lys(Z)-Gly-OBzl x Tfa)

The second step in the cationic tripeptide synthesis shown in FIG. 1 is deprotection to yield an N^{ε}-benzoxycarbonyl-protected cationic dipetide of the Formula II, in this case H-Lys(Z)-Gly-Obzl X TFA.

Compound of the formula (I) produced in Example I (Batch A-1) (Boc-Lys(Z)-Gly-OBzl) (4.2 g, 7.96 mmol) was dissolved in 15 mL of 50% TFA/DCM/15 minutes and stirred at ambient temperature over 15 minutes. The solvents were evaporated in vacuo (at approximately 40° C.) and product precipitated by addition of ethyl ether (200 mL). An oil was separated by decantation, washed with ether (3×75 mL) and dried in vacuo in the presence of NaOH in two Petrie dishes overnight to yield 3.6 g (83.5% yield) H-Lys(Z)-Gly-OBzl X TFA (Batch B-1) with calculated molecular weight: 541.54 Da (427.52+114.02) (C₂₃H₂₆N₃O₅+TFA).

Purity was estimated by TLC: R_{F}=0.27 (single spot using chloroform:methanol=9:1 (v/v)); R_{F}=0.80 (single spot using (n-butanol:acetic acid:water=4:2:2 (v/v/v)). The synthesis of H-Lys(Z)-Gly-OBzl x TFA was repeated three times (2.8 mM, 7.6 mM and 23.6 mM scale, respectively) to give 1.1 g (Batch B-2), 2.1 g (Batch B-4) and 13.4 g (Batch B-4).

### EXAMPLE 3

### Protected Tripeptide Intermediate N^{α}-Benzyloxycarbonyl-Glycine-L-Lysine (N^{ε}-Benzyloxycarbony)-Glycine Benzyl Ester (Z-Gly-Lys(Z)-Gly-OBzl)

The third step in the cationic tripeptide synthesis shown in FIG. 1, the second coupling reaction, as exemplified here to yield an N^{ε}-benzoxycarbonzyl-protected cationic tripeptide of the Formula III, in this case the novel compound, Z-Gly-Lys(Z)-Gly-OBzl.

Compond H-Lys(Z)-Gly-OBzl x TFA (II) (3.65 g, 6.65 mmol) prepared in Example II, Z-Gly-OH (1.3912 g, 6.65 mmol), HOBt x H2O (1.02 g, 6.65 mmol) and HBTU (2.522 g, 6.65 mmol) were dissolved in DMF (25 mL), stirred and cooled with an ice-bath. The NMM (2.77 mL, 25.14 mmol) was added dropwise and stirring was continued for one hour at 0° C. and overnight (19 hours) at ambient temperature (pH was controlled to keep 7.5-8 as indicated by moister pH paper). The progress of the reaction was monitored by TLC. After 19 hours of coupling, water (600 mL) was added to reaction mixture. Precipitated solid material was filtered off, washed with water (4×100 mL) and dried in vacuo in presence of P₂O₅ in two Petrie dishes to give an off-white solid: 3.65 g (Batch C-1, 89% yield) having a melting point of 104-107° C.; calculated molecular weight: 618.72 Da

The purity was estimated by TLC: R_{F}=0.56 (single spot using chloroform:methanol=9:1 (v/v) ); R_{F}=0.98 (single spot using n-butanol: acetic acid:water=4:2:2 (v/v/v)). The synthesis of Z-Gly-Lys(Z)-Gly-OBzl was repeated twice (5.9 mM and 24.7 mM) to give 3.4 g (Batch C-2) and 11.9 g (Batch C-3) of product.

### EXAMPLE 4

### Deprotection of Carboxybenzyl Protected Tripeptide to Yield Glycyl-L-Lysyl-Glycine (Gly-Lys-Gly)

The fourth step in the cationic tripeptide synthesis shown in FIG. 1, the second deprotection reaction, as exemplified here to yield an N^{ε}-benzoxycarbonzyl-protected cationic tripeptide of the Formula IV, in this case Gly-Lys-Gly. The reaction conditions are shown below.

A. Compound (III) (Batch C-1) (506.3 mg, 0.8183 mmol) was dissolved in a mixture of ethanol: acetic acid: water (8:2:1, v/v/v) (some insoluble material was noted). To the mixture, was added 10% Palladium on activated carbon (100 mg) (Aldrich) (100 mg) and the hydrogenation reaction was carried out over 1.5 hour using an initial pressure of 54 psi which dropped to 51 psi.

TLC (R_{F}=0.12 by ninhydrin and iodine visualization, and n-butanol:acetic acid:water=4:2:2 v/v/v) was used to monitor the progress of reaction. The Pd/C was filtered off using Gelman Acrodisc, LC PVDF, 0.2 mm and 10 mL syringe. The ethanol was evaporated under vacuum and the remaining solution was lyophilized to give 164 mg (77% yield) of crude product (Batch D-1); having a calculated molecular weight of 260.29; empirical formula: C₁₀H₂₀N₄O₄.

B. In a separate reactions, the protected tripeptide of Example III (C-1) (2.5 g, 4.04 mmol) was dissolved in a mixture (75 mL) of ethanol:acetic acid:water=8:2:1 (v/v/v) with stirring. After about 30 minutes, the white, solid material started to precipitate out. The DMF was added by 1 mL portions to the stirred suspension (the total volume of DMF used to dissolve precipitate was 25 mL). The 10% palladium on activated carbon (0.5 g) was added and hydrogenation was carried out over 1.5 hour (the initial pressure dropped from 55 psi to 46 psi). The Pd/C was filtered off using Gelman Acrodisc, LC PVDF, 0.2 mm and 10 mL syringe. The ethanol was evaporated under vacuo and remaining solution was diluted with water (10 mL) than lyophilized to give 1.04 g (99%) (Batch D-2).

The deprotection reaction of as described in IVB (above) was repeated twice starting from 3.4 g (5.5 mmol) and 11.9 g (19.2 mmol) of protected tripeptide (Batch C-2 and C-3) to give 1.4 g (98%) (Batch D-3) and 4.85g (96%) (D-4) of the product (Gly-Lys-Gly).

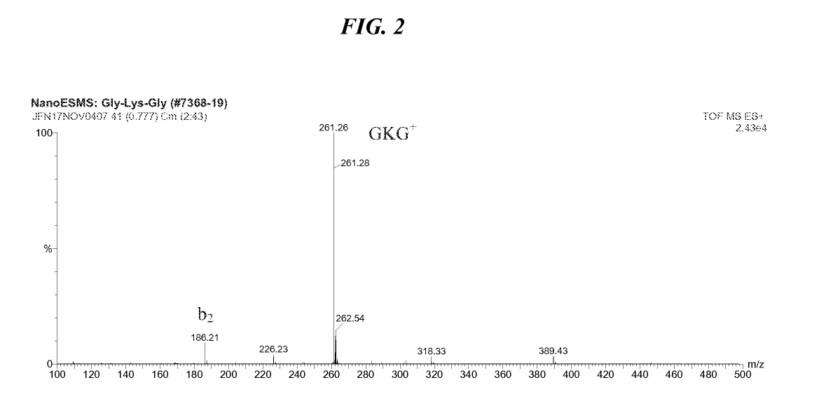

Batches D-1 (0.164 g) and D-2 (1.04 g), and D-3 (1.4 g) were dissolved in water, pooled and lyophilized yielding 2.55 g of product (D-5). The Gly-Lys-Gly (IV) (Batch D-5) was analyzed by NanoESi MS (FIG. 2):

### EXAMPLE 5

### Final Desalting and Analysis of Glycine-L-Lysine-Glycine (Gly-Lys-Gly) (IV)

For desalting, Batch D-5 (2.55 g) was dissolved in 100 mL buffer A (0.1% TFA/water) and pumped onto the two Vydac C-18 columns, 10 mm, 2.5×25 cm.

Material was eluted from the column isocratically with 100% of buffer A at a flow rate of 6 mL/min, monitoring at 220 nm and collecting 12 mL fractions. Fractions were analyzed by HPLC, pooled and lyophilized to give four fractions of Batch E given below:

| | | |
|---|---|---|
| E-1 | Yield: 1.57 g | HPLC >98% |
| E-1A | Yield: 0.47 g | HPLC >96% |
| E-1B | Yield: 0.37 g | HPLC >95% |
| E-2 | Yield: 0.04 g | HPLC = 92% |
| | 2.45 g Total | |

Fraction E-2 was a DMF extracted impurity from a syringe that strongly absorbs at 280 nm. Based on this finding the use of glass filter and not Gelman Acrodisc for filtration is recommended.

Batch D-4 of Gly-Lys-Gly (GKG) (4.85 g) was dissolved in buffer A and desalted by HPLC in a similar manner. Fractions were pooled and lyophilized. Three lots of the product were produced as follows: GKG-A (0.092 g), GKG-B (5.19 g), and GKG-C (1.8 g); Empirical Formula C₁₀H₂₀N₄O₄, M.W. 260.2953 Da (monoisotopic M.W.: 260.14832 Da).

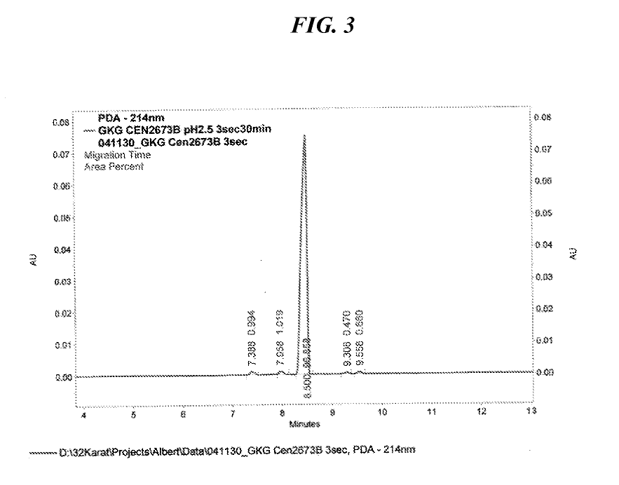

The observed M.W. by infusion ES-MS was 261.27 Da and by Nano ES-MDS was 260.29 Da. The purity by capillary electrophoresis (CE): 98%-95% and by TLC=100% [n-butanol:acetic acid:water=4:2:2 (v/v/v); R_{F}=0.12 (single spot using ninhydrin, iodine)] and 100% isopropanol:ammonia (27%)=1:1 (v/v); R_{F}=0.2 (single spot using ninhydrin, iodine)]. FIG. 3 shows CE of the Gly-Lys-Gly (GKG-B) (Beckman Coulter P/ACETM MDQ instrument).

### EXAMPLE 6

### Biological Activity of Gly-Lys-Gly

Two preparations of the tri-peptide, Gly-L-Lys-Gly, were compared for their effect on growth and productivity of a transfectoma cell line which is an SP2/0 derived line engineered to produce an anti-cytokine monoclonal antibody (mAb). The final lot of GKG prepared in Example V was compared to commercial prepared GKG purchased from Bachem (King of Prussia, Pa.).

Cells were cultured in a proprietary defined medium, MET1.5, supplemented with 7.0 mM glutamine, 10 g/L MHX, and 2.0 g/L sodium bicarbonate. The Centocor-made GKG peptide in quantities of 0.35 g/L, 0.70 g/L, and 1.40 g/L were added to 250 mL of MET 1.5 media. The GKG from Bachem was added to 250 mL of MET 1.5 media at concentrations of 0.38 g/L, 0.76g/L, and 1.5 g/L. The cells were cultured in 125 mL shaker flasks and were rotating at 125 RPM. The culture was also incubated at 37° C. in a humidified atmosphere with 5% CO₂ and 95% air. The culture volume was 40 mL. Two control flasks and two flasks per peptide concentration were sampled at days zero, one, two, three, four, five, and six for viable cell density and antibody determination.

The cultures were inoculated at a density of 2×10⁵ cells/mL and were passaged three times (split every three days). On the third passage they were incubated until the decline phase.

Viable cells and dead cells were calculated by trypan exclusion with the Cedex cell counter. The mAb concentrations were determined by immunoturbidity via the IMMAGE 800 nephelometer (Beckman, Coulter, Brea, Calif.). The effects of the Gly-Lys-Gly peptide on antibody secretion by a production cell line was gauged by calculating integral viable density (IVCC), and titer. IVCC was calculated by integrating the areas under the growth curves. Specific antibody productivity was calculated as the antibody production per day (titer) divided by IVCC.

Results

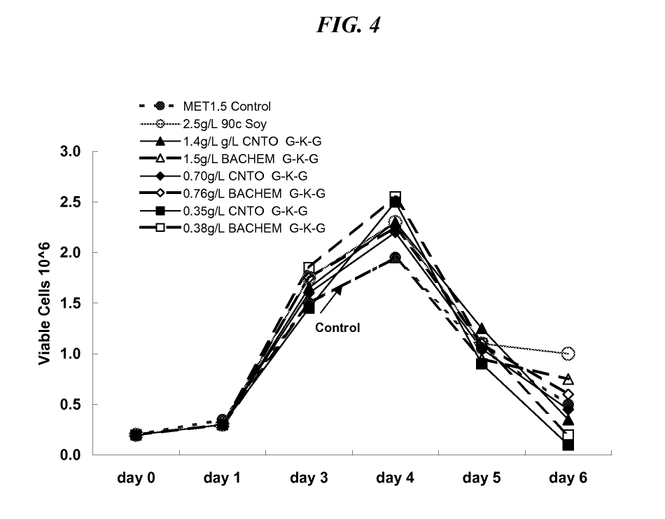

Peak viable cell densities were increased when both the Centocor and Bachem-made-peptide was added at a concentration of 0.35 g/L and 0.38 g/L, respectively (FIG. 4). The addition of 0.35 g/L of the Centocor-made peptide resulted in a viable cell density increase from to 2.0×10⁶ viable cells/mL (unsupplemented control) to 2.5×10⁶ viable cells/mL (FIG. 3). The addition of 0.38 g/L of the Bachem-made-peptide raised the viable cell density to 2.6×10⁶ viable cells/mL. Each point is the mean of results from two samples.

Supplementation of a proprietary chemically defined medium, MET1.5, with 1.4 g/L and 1.5 g/L of Gly-Lys-Gly made by Centocor and Bachem respectively resulted in 21% and 34% increases in titer, respectively as shown in FIG. 4 by the concentration of mAb (mg/ml). Viable cell density and percent viability increases could not be observed at this concentration.

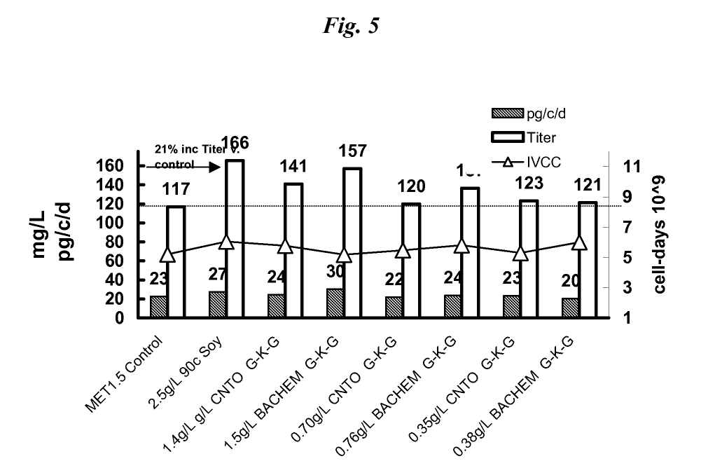

No enhancement of the product yield or viable cell density was found when 0.7 g/L of the Centocor-made-peptide was added. The titer rose from 117 mg/L to 137 mg/L when MET1.5 was supplemented with 0.76 g/L of the Bachem-made-peptide (FIG. 5). Each point is the mean of results from two samples.

In a previous experiment, a proprietary chemically defined Chinese Hamster Ovary (CHO) medium was supplemented with both the Centocor and Bachem-made peptides at the same concentrations above. There was minimal to no effect on growth and productivity of the CHO line tested (results not shown).

What is claimed:

1. A method of solution phase peptide synthesis for producing a cationic tripeptide of the formula Gly-Xaa-Gly wherein Xaa is a basic alpha amino acid having one to two basic sidechain functional groups and a free amino group at the alpha carbon selected from the group consisting of Lys, Orn, His, Arg or Homoarg, Dab, or Dap, comprising:
a) reacting Xaa wherein said basic sidechain functional groups are protected by a benzyloxycarbonyl moiety (A) and the alpha amine by an orthogonal protecting group (B), said orthogonal protecting group being labile to hydrolysis under conditions wherein the benzyloxycarbonyl-sidechain protection is not labile, with Gly or a carboxyl derivative (Gly-X) to get a compound of the formula B-Xaa(A)ₙ-Gly-X,
i) wherein A is selected from the group consisting of benzyloxycarbonyl, 4-chlorobenzyloxycarbonyl, and 2-chlorobenzyloxycarbonyl;
ii) wherein n=1 when Xaa is Lys, Orn, His, Dap or Dab and n=2 when Xaa is Arg or HomoArg; and
iii) wherein X may be OH, O-Bzl, O-R₁ or —N(R₁R₂) where R₁ and R₂ are independently H, small alkyl, acyl, fatty acyl, or aryl;
b) removing the protecting group at the alpha amine of Xaa using suitable deprotecting conditions;
c) reacting the deprotected compound with benzyloxycarbonyl protected Gly (A-Gly) to get a compound of formula N^{α}-A-Gly-Xaa(A)n-Gly-X;
d) removing all benzyloxycarbonyl moieties using suitable deprotection conditions under neutral conditions; and
e) purifying the tripeptide Gly-Xaa-Gly-X.

2. The method of solution phase peptide synthesis according to claim 1 wherein Xaa is Lys and the compound of step c is N^{α}-benzyloxycarbonyl-Gly-L-lys (N^{ε}-benzyloxycarbonyl)-Gly benzylester.

3. A compound of the formula: N^{α}-A-Gly-Xaa(A)ₙ-Gly or N^{α}-A-Glycine-L-Xaa(A)ₙ-Gly-X wherein:
i) A is selected from the group consisting of benzyloxycarbonyl, 4-chlorobenzyloxycarbonyl, and 2-chlorobenzyloxycarbonyl;
ii) Xaa is selected from Lys, Orn, His, Arg or Homoarg, Dab, or Dap;
iii) n=1 when Xaa is Lys, Orn, His, Dap or Dab and n=2 when Xaa is Arg or HomoArg; and
iv) X is selected from OH, O-Bzl, O-R₁ or —N(R₁ R₂) where R₁ and R₂ are independently H, small alkyl, acyl, fatty acyl, or aryl.

4. The process according to claim 1 wherein the final deprotection reaction of step (d) is selected from (a) catalytic hydrogenolysis using molecular hydrogen and palladium (H₂/Pd) or catalytic transfer hydrogenolysis; (b) reduction with metallic sodium in liquid ammonia and (c) reaction with strong acids using halogenated acids or hydrohalogenic acids in acetic acid (HBr/AcOH) or liquid HF, BBr₃/DCM, TFA/thioanisole and sulfonic acids.

5. The process of claim 4 wherein the final deprotection reaction of step (d) is performed using catalytic hydrogenolysis with molecular hydrogen and palladium (H₂/Pd) or catalytic transfer hydrogenolysis.

6. The process of claim 1 wherein the orthogonal protecting group (B) is selected from the group consisting of Boc, Bpoc, 1-Adoc, Ddz, Fmoc, Nsc, and Msc.

7. A method of solution phase peptide synthesis of producing a cationic tripeptide of the formula Gly-Xaa-Gly-NH₂ wherein Xaa is an amino acid having one or more basic side chain functional groups selected from the group consisting of Lys, Orn, His, Arg or Homoarg, Dab, or Dap, comprising
a) reacting a Xaa wherein the basic sidechain functional groups are protected by a carbonylbenzoxy group moiety (A) and the alpha amine by an orthogonal protecting group (B), said orthogonal protecting group being labile to hydrolysis under conditions wherein the benzyloxycarbonyl-sidechain protection is not labile, with Gly-NH₂ to get a compound of the formula Boc-Xaa(A)ₙ-Gly-NH₂;
i) wherein A is selected from the group consisting of benzyloxycarbonyl, 4-chlorobenzyloxycarbonyl, and 2-chlorobenzyloxycarbonyl;
ii) wherein n=1 when Xaa is Lys, Orn, His, Dap or Dab and n=2 when Xaa is Arg or HomoArg; and
b) deprotecting the alpha amine of Xaa using a solution comprising TFA;
c) reacting the deprotected compound (I) with benzyloxycarbonyl protected Gly (A-Gly) to get a compound of formula N^{α}-Benzyloxycarbonyl-Glycine-L-Xaa(Z)-Glycine amide;
d) deprotecting all amines using hydrogenation; and
e) purifying the tripeptide Gly-Lys-Gly.
